(19) United States
(12) Patent Application Publication
(10) Pub. No.: US 2009/0156985 A1
(43) Pub. Date: Jun. 18, 2009

(54) Ophthalmic Surgical Cassette and System

(76) Inventors: Matt D. Hottmann, Larsen, WI (US); MacIntosh E. Perry, Garner, NC (US); Corey E. Frye, Fuquay Varina, NC (US); Jonathan T. Walter, Wake Forest, NC (US)

Correspondence Address: Bausch & Lomb Incorporated, One Bausch & Lomb Place, Rochester, NY, 14604-2701, US

(21) Appl. No.: 11/957,651

(22) Filed: Dec. 17, 2007

(51) Int. Cl. A61M 27/00; A61F 9/007

(52) U.S. Cl. 604/35

(57) ABSTRACT

An ophthalmic surgical system 10 for aspirating fluids and tissue away from a patient's eye comprising a surgical handpiece 12, a cassette 14, and a control console 16. Ophthalmic cassette 14 includes a first cassette portion 20 for attachment to a pump 24, a second cassette portion 22 for attachment to the surgical handpiece 12 and a data storage device 18 attached to cassette 14. Control console 16 includes the pump 24, a data storage device interface 26 and a processing device 28.

### BACKGROUND OF THE INVENTION

1. Field of the Invention

The present invention is directed towards an ophthalmic surgical cassette and system. More specifically, the present application is directed towards an ophthalmic surgical cassette comprising a data storage device.

2. Description of Related Art

During ophthalmic surgical procedures, excised tissue and aspirant fluids are collected in an ophthalmic surgical cassette, usually located remote from a surgical handpiece. Ophthalmic surgical cassettes generally include an interior volume for collection of tissue and fluid from the surgical site for later disposal. These cassettes may be disposable or reusable. A reusable cassette is typically flushed clean after use and is then sterilized by autoclaving.

As with any piece of surgical equipment, it is of great importance that only operable cassettes be used for surgery. Thus, it may be preferable to dispose of a cassette after a certain number of uses or after a certain time period of use. By providing a way to automatically identify the number of times a particular cassette has been utilized, manual recording of such information could be eliminated. There are known systems for providing interaction between a cassette and a console system, such as those found in U.S. Pat. Nos. 6,036,458 and 6,059,544.

U.S. Pat. No. 6,036,458 discusses an ophthalmic cassette with a bar code element disposed on the cassette and a bar code reader on a control console for providing an active interface between the cassette and the console. The bar code provides for communication of cassette characteristics, such as serial number and history of the cartridge.

U.S. Pat. No. 6,059,544 mentions a surgical cassette that contains a series of identifying bar codes that can be optically scanned by a surgical instrument for cassette identification.

Although these systems can record the history of the cassettes, the information is not stored on the cassette, but in the console or computer. Therefore, there exists a need for an improved ophthalmic surgical cassette and system.

### BRIEF DESCRIPTION OF THE DRAWINGS

FIG. 1 is a graphical illustration of an ophthalmic surgical system; and

FIG. 2 is a graphical illustration of an ophthalmic surgical cassette.

### DETAILED DESCRIPTION

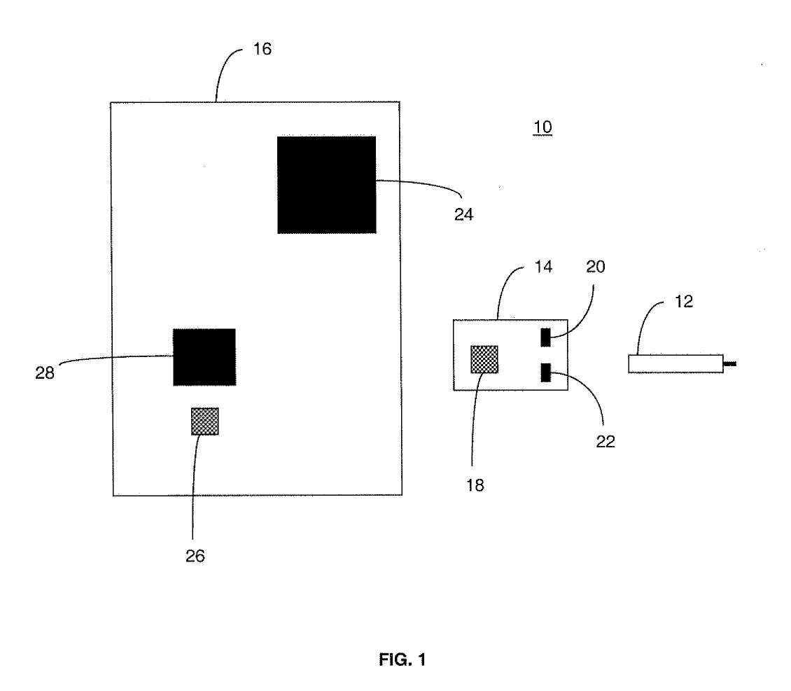

The present invention provides an ophthalmic surgical system 10 for aspirating fluids and/or tissue away from a patient's eye. FIG. 1 shows a graphical representation of an ophthalmic surgical system 10 in accordance with the present invention. System 10 includes a surgical handpiece 12, a cassette 14, which will be described in greater detail hereinafter and a control console 16.

Examples of surgical handpieces useful with the present invention include ophthalmic probes and handpieces (e.g., vitrectomy cutters and phacoemulsification handpieces).

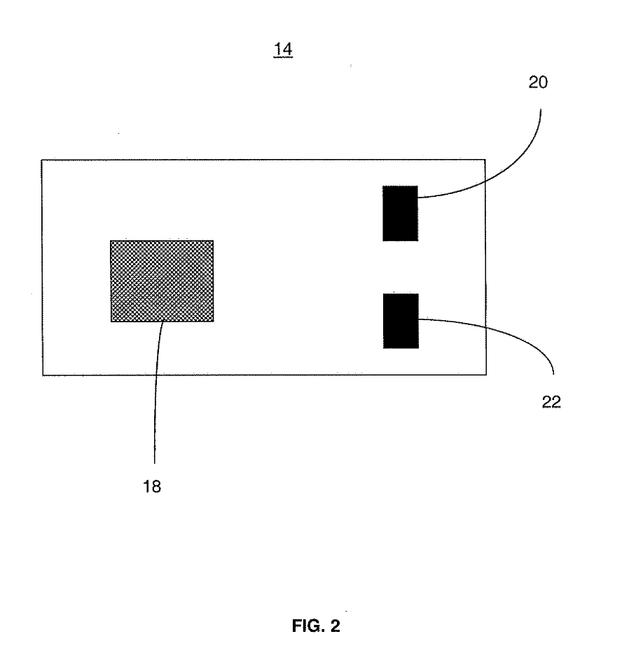

The present invention further provides an ophthalmic surgical cassette 14 for use with a pump for collecting aspirant fluid and tissue from a patient's eye. FIG. 2 shows a graphical illustration of cassette 14, in accordance with the present invention. Cassette 14 includes an attached data storage device 18, a first portion 20 for attachment to a pump 24, a second portion 22 for attachment to the surgical handpiece 12, wherein the cassette 14 is configured for working with the pump 24 and receiving aspirant fluid and/or tissue from surgical handpiece 12. Though cassette 14 is shown apart from console 16, those skilled in the art will realize that cassette 14 is typically held on or within console 16 during operation. Other than data storage device 18, cassette 14 may be any known ophthalmic cassette.

Data storage device 18 preferably comprises non-volatile memory. Non-volatile memory may be memory that can retain stored information even when not powered. Thus, data storage device 18 does not require its own power source. Data storage device 18 is preferably powered by control console 16 through known means.

Electrically erasable programmable read-only memory (EEPROM) is an example of non-volatile memory and has the advantage of being able to selectively erase any part of the chip without the need to erase the entire chip. Therefore, stored information may be updated and then retained when external power is removed.

Data storage device 18 should be protected against tampering. Secure EEPROM provides authentication and encryption protocols in order to provide secure communications and data storage between the device 18 and console 16.

Data storage device 18 may utilize contact and/or contactless memory. Contactless devices are enabled to send and receive wireless communications. Conversely, contact devices are enabled to send and receive direct contact communications. An example of direct contact communication is an electrically coupled signal (e.g., a contact chip inserted in an acceptor device where pins attached to a reader make contact with pads to read information on the data storage device).

Data storage device 18 may comprise a smart chip. Smart chips contain microprocessors and memory. Smart chips are often embedded into plastic cards known as smart cards. Therefore, data storage device 18 may comprise a smartcard format. Smartcards are available from a variety of manufacturers, over a range of storage capabilities.

Data storage device 18 can be glued into a mechanical depression in cassette 14 or could be molded into place.

Data storage device 18, when attached to a reusable cassette which must be autoclaved between uses, must be autoclave compatible.

Data storage device 18 of the present invention preferably stores and adjusts at least one parameter. These parameters include any information stored on the data storage device which relate to the characteristics of the cassette and/or the cassette usage history. The at least one parameter may be stored in the device during manufacture of cassette 14 or in response to surgical use of cassette 14. Therefore, information may be stored permanently on the device and may not be modified, while other information may be adjusted or re-written throughout the lifetime of the cassette, often in response to cassette usage.

Examples of cassette characteristics include whether the cassette is disposable or reusable, a cassette serial number, manufacturer (authenticity) and intended surgical procedure. Along with the intended surgical procedure, parameters such as aspiration vibration frequency of a surgical handpiece, vacuum pressure, aspiration fluid flow rate, pump speed, irrigation fluid pressure and irrigation fluid flow rate may be preprogrammed for a specific surgical procedure. These parameters may represent default parameters suitable for a particular surgical procedure which may be manually adjusted by the surgeon. Similarly, information stored on the data storage device may function to prevent various functions based upon the cassette configuration and thus, prevent use of a cassette in an unauthorized manner. In this way, data storage device 18 attached to cassette 14 stores data indicative of parameters of the cassette or use of the cassette during a life cycle of the cassette

Parameters stored on the device following manufacture or in response to cassette usage may be permanently stored or adjustable during a life cycle of the cassette. Examples of such parameters include the number of times the particular cassette has been used, the time period of each use, and the surgical conditions during each use.

Console 16 includes a pump 24, which may provide a source of suction for the aspirating function of surgical handpiece 12, a data storage device interface 26 and a processing device 28. During surgical use of ophthalmic system 10, the various components are often physically connected. For example, cassette 14 may be coupled to pump 24 and to surgical handpiece 12.

Data storage device interface 26 and processing device 28 may be configured for communication with the cassette data storage device 18. Specifically, information stored on data storage device 18 is read and adjusted by processing device 28 via interface 26. When a contact data storage device is used, data storage device 18 must be in contact with data storage device interface 26. Therefore, data storage device interface 26 may be used as a communication medium between data storage device 18 and processing device 28. Thus, data stored in data storage device 18 is capable of being adjusted during the life cycle of the cassette.

Because data storage device 18 is physically attached to cassette 14, important information regarding cassette 14 and its surgical history is automatically and reliably available on cassette 14. Therefore, such information may be accessed by any console or system with which cassette 14 and the attached data storage device 18 can interact.

Thus, has been shown an inventive ophthalmic surgical cassette and system, variations of which will be apparent to those skilled in the art. Such variations are intended to be within the scope of the present invention and the claims set forth herein.

We claim:

1. An ophthalmic surgical cassette for use with a pump for collecting aspirant fluid and tissue from a patients eye, the cassette comprising:
a first cassette portion for attachment to a pump;
a second cassette portion for attachment to a surgical handpiece;
a data storage device attached to the cassette for storing data indicative of parameters of the cassette or use of the cassette during a life cycle of the cassette; and
wherein the data stored in the data storage device is capable of being adjusted during the life cycle of the cassette.

2. The cassette of claim 1, wherein the data storage device stores at least one parameter.

3. The cassette of claim 1, wherein the data storage device comprises electrically erasable programmable read-only memory (EEPROM).

4. The cassette of claim 1, wherein the data storage device comprises a smart chip.

5. The cassette of claim 1, wherein the data storage device comprises a processing device.

6. The cassette of claim 1, wherein the at least one parameter is permanently stored in the data storage device.

7. The cassette of claim 1, wherein the at least one parameter defines the usable life of the cassette

8. The cassette of claim 1, wherein the at least one operating parameter is stored in response to cassette usage.

9. The cassette of claim 1, wherein the at least one parameter includes total number of uses, total time period of each use and/or other surgical history information.

10. An ophthalmic surgical system for aspirating fluids and tissue away from a patient's eye comprising:
a surgical handpiece;
a cassette, the cassette including a first cassette portion for attachment to a pump and a second cassette portion for attachment to a surgical handpiece;
a data storage device attached to the cassette, wherein the data storage device is capable of being adjusted during a life cycle of the cassette;
a control console, the control console including a pump, a data storage device interface and a processing device for adjusting the data stored in the data storage device; and
wherein the cassette is configured for receiving aspirant fluid and tissue from a surgical handpiece.

11. The system of claim 10, wherein the processing device and data storage device interface are configured for communication with the data storage device.

12. The system of claim 10, wherein the cassette is coupled to the surgical handpiece and to the control console.

13. The system of claim 11, wherein the communication includes reading at least one parameter stored in the data storage device.

14. The system of claim 11, wherein the communication includes writing to the data storage device.
